(19) United States
(12) Patent Application Publication
(10) Pub. No.: US 2023/0255878 A1
(43) Pub. Date: Aug. 17, 2023

(54) COLOR CHANGING COSMETIC ADHESIVES

(71) Applicant: Leah Shoats, Monument, CO (US)

(72) Inventor: Leah Shoats, Monument, CO (US)

(21) Appl. No.: 17/673,066

(22) Filed: Feb. 16, 2022

(51) Int. Cl. A61K 8/9794; A61K 8/9789; A61K 8/81; A61K 8/37; A61Q 1/10

(52) U.S. Cl. CPC A61K 8/9794; A61K 8/37; A61K 8/8147; A61K 8/9789; A61Q 1/10; A61K 2800/438

(57) ABSTRACT

Color changing cosmetic adhesives for adhering a cosmetic i tem. The color changing cosmetic adhesives include an adhesive formulation and a dye formulation. The dye formulation is distributed within the adhesive formulation. The dye formulation is configured to impart a first color to the adhesive formulation while the adhesive formulation is stored in a container. The dye formulation is configured to impart a second color to the adhesive formulation after the adhesive formulation is applied to skin. In some examples, the color changing cosmetic adhesive includes a preservative formulation.

### BACKGROUND

The present disclosure relates generally to cosmetic adhesives. In particular, color changing cosmetic adhesives for adhering items to skin are described.

False eyelashes are a popular cosmetic accessory for fashion and personal style. False eyelashes are worn by adhering them to eyelids with a cosmetic adhesive. However, known cosmetic adhesives for adhering items to skin are not entirely satisfactory.

For example, existing cosmetic adhesives do not provide an indication when the adhesive is sufficiently tacky to adhere a cosmetic item. It would be desirable for cosmetic adhesives to provide an indication when the adhesive was ready to adhere a cosmetic item. It would be particularly helpful for a cosmetic adhesive to provide a visual indication that the adhesive was sufficiently tacky to adhere a cosmetic item like a false eyelash.

Thus, there exists a need for cosmetic adhesives that improve upon and advance the design of known cosmetic adhesives. Examples of new and useful color changing cosmetic adhesives relevant to the needs existing in the field are discussed below.

### SUMMARY

The present disclosure is directed to color changing cosmetic adhesives for adhering a cosmetic item. The color changing cosmetic adhesives include an adhesive formulation and a dye formulation. The dye formulation is distributed within the adhesive formulation. The dye formulation is configured to impart a first color to the adhesive formulation while the adhesive formulation is stored in a container. The dye formulation is configured to impart a second color to the adhesive formulation after the adhesive formulation is applied to skin. In some examples, the color changing cosmetic adhesive includes a preservative formulation.

### BRIEF DESCRIPTION OF THE DRAWINGS

FIG. 1 is a view of a color changing cosmetic adhesive initially applied to an eyelid.

FIG. 2 is a view of the color changing cosmetic adhesive shown in FIG. 1 transitioned to a second color on the eyelid.

FIG. 3 is a view of a false eyelash adhered to the color changing cosmetic adhesive shown in FIG. 1 on the eyelid.

FIG. 4 is a schematic view of the color changing cosmetic adhesive shown in FIG. 1.

FIG. 5 is a flow diagram of a method of making the color changing cosmetic adhesive shown in FIG. 1.

### DETAILED DESCRIPTION

The disclosed color changing cosmetic adhesives will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various color changing cosmetic adhesives are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

Composition percentages are expressed as a weight percent of the overall color changing cosmetic adhesive formulation unless otherwise indicated.

### Color Changing Cosmetic Adhesives

With reference to the figures, color changing cosmetic adhesives will now be described. The color changing cosmetic adhesives discussed herein function to adhere cosmetic items to a person’s skin. In the particular example shown in the figures, the color changing cosmetic adhesive functions to adhere a false eyelash to a person’s eyelid.

The reader will appreciate from the figures and description below that the presently disclosed color changing cosmetic adhesives address many of the shortcomings of conventional cosmetic adhesives. For example, the novel color changing cosmetic adhesives described below provide an indication when the adhesive is ready to adhere a cosmetic item. In particular, the novel color changing cosmetic adhesives described herein provide a visual indication that the adhesive is sufficiently tacky to adhere a cosmetic item, such as a false eyelash.

### Contextual Details

Ancillary features relevant to the color changing cosmetic adhesives described herein will first be described to provide context and to aid the discussion of the color changing cosmetic adhesives.

### Cosmetic Item

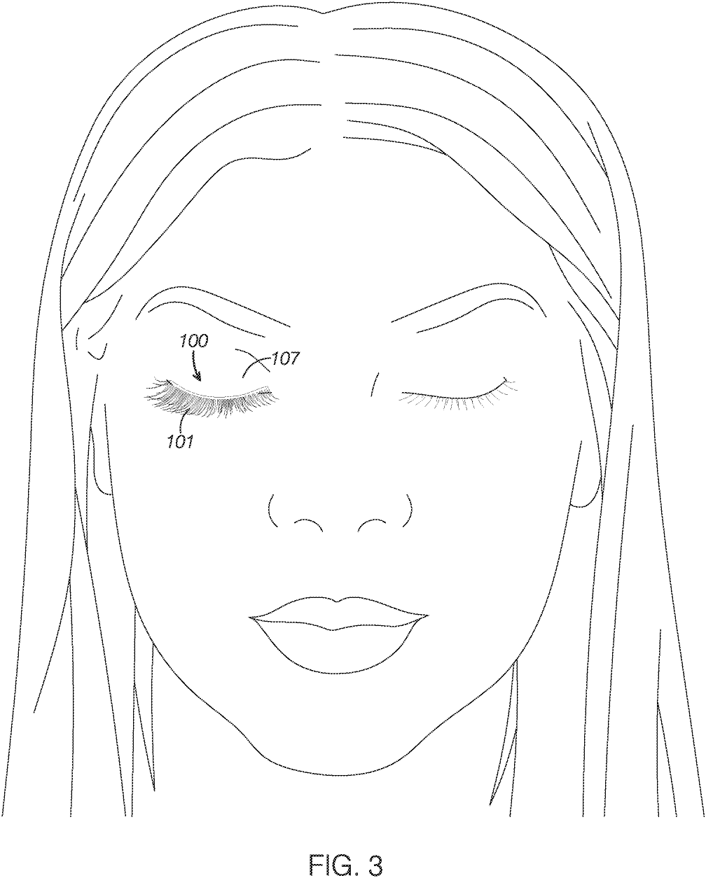

The color changing cosmetic adhesives described in this document function to adhere a cosmetic item, such as cosmetic item 101 shown in FIG. 3. As shown in FIG. 3, cosmetic item 101 is a false eyelash.

However, the cosmetic item may be any currently known or later developed type of cosmetic item. The reader will appreciate that a variety of cosmetic item types exist and could be used in place of the cosmetic item shown in the figures. In addition to the types of cosmetic items existing currently, it is contemplated that the color changing cosmetic adhesives described herein could be used to adhere new types of cosmetic items developed in the future.

The size and shape of the cosmetic item may be varied as needed for a given application. In some examples, the item is larger relative to the person depicted in the figures. In other examples, the item is smaller relative to the person than depicted in the figures. Further, the reader should understand that the item and the person may all be larger or smaller than described herein while maintaining their relative proportions.

### Container

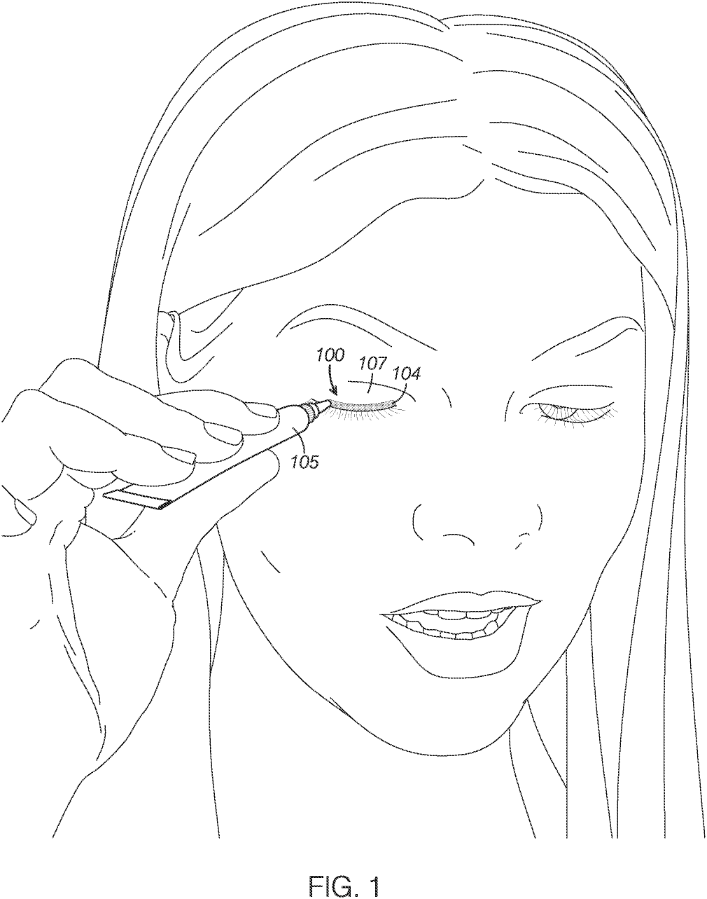

The color changing cosmetic adhesives described herein may be stored in a sealable container, such as container 105 shown in FIG. 1. The container may be any currently known or later developed type of container. The reader will appreciate that a variety of container types exist and could be used in place of the container shown in the figures. In addition to the types of containers existing currently, it is contemplated that the color changing cosmetic adhesives described herein could be stored in new types of containers developed in the future.

The number of containers used to store the color changing cosmetic adhesive may be selected to meet the needs of a given application. The reader should appreciate that the number of containers may be different in other examples than is shown in the figures. For instance, some examples include additional containers than described in the present example.

The size and shape of the container may be varied as needed for a given application. In some examples, the container is larger relative to the person than depicted in the figures. In other examples, the container is smaller relative to the person than depicted in the figures. Further, the reader should understand that the sealed container and the person may all be larger or smaller than described herein while maintaining their relative proportions.

### Color Changing Cosmetic Adhesive Embodiment One

With reference to FIGS. 1-5, a color changing cosmetic adhesive 100 will now be described as a first example of a color changing cosmetic adhesive. The reader can see in FIGS. 1-3 that color changing cosmetic adhesive 100 is for adhering a cosmetic item 101 to skin.

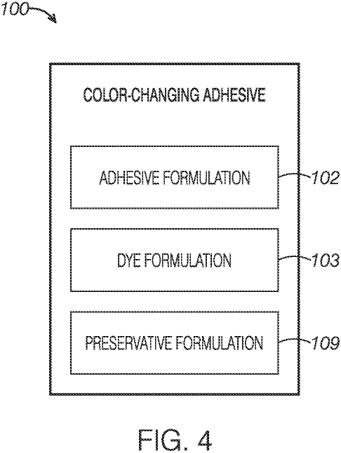

As depicted in FIG. 4, color changing cosmetic adhesive 100 includes an adhesive formulation 102, a dye formulation 103, and an optional preservative formulation 109. In other examples, the color changing cosmetic adhesive includes fewer components than depicted in the figures, such as not including a preservative formulation. In certain examples, the color changing cosmetic adhesive includes additional or alternative components than depicted in the figures.

### Adhesive Formulation

Adhesive formulation 102 functions to adhere cosmetic item 101 to a person’s skin. With reference to FIGS. 1-3, adhesive formulation 102 is applied to an eyelid 107. As shown in FIG. 3, adhesive formulation 102 is sufficiently cohesive to adhere cosmetic item 101 when adhesive formulation 102 cures to a threshold extent.

Adhesive formulation 102 is configured to cure when adhesive formulation 102 is exposed to air humidity. As depicted in FIGS. 1-3, adhesive formulation 102 cures to the threshold extent necessary to adhere cosmetic item 101 when adhesive formulation 102 is exposed to air humidity for a threshold duration. The threshold duration of the adhesive formulation may depend on the pH level of the eyelid. Adhesive formulation 102 is tacky when cured to the threshold extent

Adhesive formulation 102 has an initial pH level when stored in sealed container 105. The initial pH level of adhesive formulation 102 is between 7.16 and 8.16 and preferably between 7.5 and 7.7. Other initial pH level ranges may be selected.

The pH level of adhesive formulation 102 decreases from the initial pH level to a new, current pH level when applied to skin. The current pH level of adhesive formulation 102 is between 5.4 and 7.15 when dye formulation 103 imparts second color 106 to adhesive formulation 102.

Adhesive formulation 102 is comprised of adhesive ingredients. The adhesive ingredients comprise water, a copolymer of acrylates and ethylhexyl acrylate, glycerin, butyl methacrylate, PEG-40 hydrogenated castor oil, tocopheryl acetate, panthenol, biotinoyl tripeptide-1, and sodium dehydroacetate. In other examples, the adhesive formulation comprises different adhesive ingredients.

The adhesive ingredients may comprise a range of compositions. One particular composition of the adhesive ingredients is 41.2% water, 50.4% acrylates and ethylhexyl acrylate copolymer, 3.2% glycerin, 2.0% butyl methacrylate, 1.0% PEG-40 hydrogenated castor oil, 0.3% tocopheryl acetate, 0.02% panthenol, 0.00012% biotinoyl tripeptide-1, and 0.46% sodium dehydroacetate. The reader should understand that different composition ranges for the adhesive ingredients are contemplated.

### Dye Formulation

The role of dye formulation 103 is to impart color to adhesive formulation 102. More specifically, dye formulation 103 serves to change the color of color changing cosmetic adhesive 100 once the adhesive is applied to a person’s skin. As shown in FIG. 4, dye formulation 103 is distributed within adhesive formulation 102.

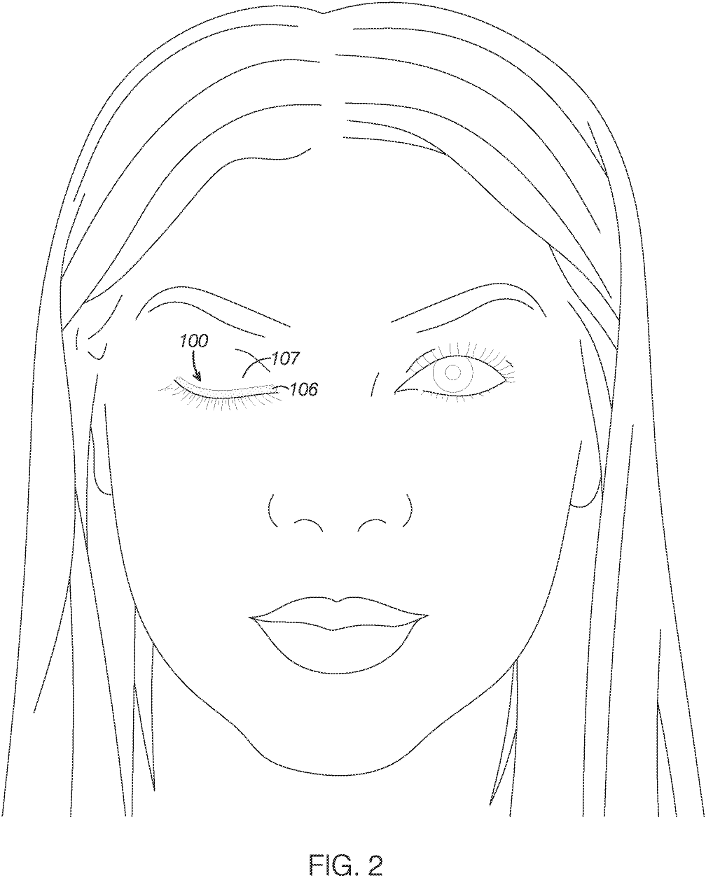

The reader can see in FIG. 1 that dye formulation 103 is configured to impart a first color 104 to adhesive formulation 102 while adhesive formulation 102 is stored in a sealed container 105. As depicted in FIG. 2, dye formulation 103 is configured to impart a second color 106 to adhesive formulation 102 after adhesive formulation 102 is applied to skin.

Dye formulation 103 imparts second color 106 to adhesive formulation 102 when a current pH level of adhesive formulation 102 decreases from the initial pH level. Dye formulation 103 includes anthocyanins, which are substances that change color in response to pH changes. Anthocyanins are naturally occurring substances called flavonoids found in different plants. Anthocyanins change color at different pH levels because their molecular structure shifts as the pH of the solution they are in changes from acidic to basic and vice versa.

For example, at a low pH of around 3, the anthocyanin molecule is protonated, which means the phenolic —OH groups have hydrogens. In this environment, the anthocyanin is a positive ion, also known as a cation. The anthocyanin absorbs light in the blue-green spectrum (approx. 450-560 nm) and appears red to the human eye.

As the pH of the environment increases, however, the anthocyanin molecules become deprotonated, which means that protons are removed from the phenol groups. As the anthocyanins depronate the light absorption of the molecule shifts to absorb light in the yellow-orange spectrum (approx. 570-620 nm) giving it a blue-ish purple appearance to the human eye.

Dye formulation 103 includes a vegetable-based ingredient. In particular, dye formulation 103 includes a purple carrot extract. In other examples, the dye formulation includes additional or alternative ingredients with anthocyanins. For example, the dye formulation may a grain-based ingredient, such as a purple rice extract.

### Colors

The reader can see in FIGS. 1 and 2 that first color 104 absorbs light in a first wavelength range and that second color 106 absorbs light in a second wavelength range. In the particular example shown in the figures, the first wavelength range is 570 to 620 nanometers and the second wavelength range is 450 to 560 nanometers. However, the wavelength ranges may be different in different examples.

In general, the wavelength ranges for the first and second colors may be selected to be any ranges that are sufficiently different to be visually distinct from each other. For examples, the colors may change from a light color to a dark color or from a dark color to a light color.

### Preservative Formulation

Preservative formulation 109 functions to extend the shelf-life of color changing cosmetic adhesive 100. However, the preservative formulation is optional. In certain examples, the color changing cosmetic adhesive does not include a preservative formulation.

In one example, preservative formulation 109 includes phenoxyethanol and ethylhexylglycerin. A particular composition of the preservative ingredients is 0.9% phenoxyethanol and 0.1% ethylhexylglycerin. In other examples, the preservative formulation comprises different preservative ingredients and/or different composition percentages.

### Overall Color Changing Cosmetic Adhesive Parameters

The color changing cosmetic adhesive may be formulated to meet certain specifications or parameters. For example, the viscosity of the color changing cosmetic adhesive may be selected to be between 39,000 to 41,000 cP. The density of the color changing cosmetic adhesive may be formulated to be between 1.06 to 1.18. The initial pH level of the color changing cosmetic adhesive is formulated to be between 7.16 and 8.16 and preferably between 7.5 and 7.7.

### Method of Making Color Changing Cosmetic Adhesive

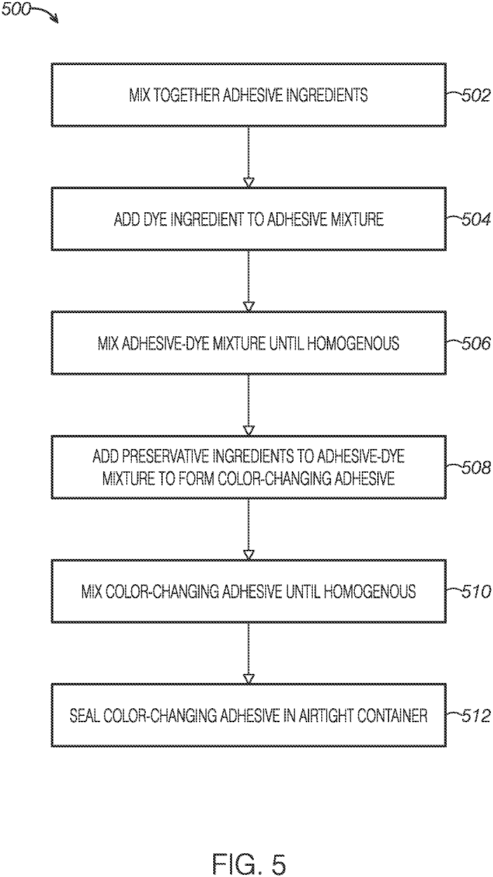

With reference to FIG. 5, a method of making color changing cosmetic adhesive 100, method 200, will be described. As shown in FIG. 5, method 500 includes six steps; however, different method examples may include fewer, additional, or alterative steps than shown in FIG. 5.

Step 502 of method 500 is mixing together adhesive ingredients. The adhesive ingredients may be the adhesive ingredients described above and in the quantities described above. Mixing at step 502 includes adding the adhesive ingredients one at a time starting with acrylates and ethylhexyl acrylate copolymer, then water, and then sodium dehydroacetate. The adhesive mixture is mixed at between 200 to 400 rpm at room temperature until the ingredients are homogenous.

At step 504, dye ingredients are added to the adhesive mixture formed in step 502. The dye ingredients may include purple carrot extract and/or purple rice extract among other ingredients with anthocyanins. The dye ingredient quantities may correspond to the dye ingredient quantities described above.

Step 506 of method 500 includes mixing the dye ingredients with the adhesive mixture until a homogenous adhesive-dye mixture is formed. Mixing the ingredients at step 506 may be conducted at 200 to 400 rpm at room temperature. Step 506 may be performed concurrently and/or after step 504.

At step 508, preservative ingredients are added to the adhesive-dye mixture. The preservative ingredients may be the preservative ingredients described above, including phenoxyethanol and ethylhexylglycerin, and in the quantities described above. After the preservative ingredients are added to the adhesive-dye mixture at step 508, a color changing cosmetic adhesive is formed.

At step 510, the color changing cosmetic adhesive is mixed until the ingredients are homogenous. Mixing at step 510 may occur at between 200 to 400 rpm at room temperature until the ingredients are homogenous.

After a homogenous color changing cosmetic adhesive is mixed at step 510, it may be sealed in an airtight container at step 512. Any currently known or later developed technique for sealing the adhesive in a container may be used.

In one example, 5.0 grams of purple rice extract was mixed homogenously with 95 grams of an adhesive formulation prepared according the method, ingredients, and composition parameters described above. The resulting color changing cosmetic adhesive turned from an opalescent blue to a dark purple when becoming tacky. When the color changing cosmetic adhesive had completely dried it became the darkest shade of plum purple.

In another example, 1.0 gram of purple rice extract was mixed homogenously with 99 grams of an adhesive formulation prepared according the method, ingredients, and composition parameters described above. The resulting color changing cosmetic adhesive turned from a pastel periwinkle purple blue to a translucent pink when becoming tacky. When the color changing cosmetic adhesive had completely dried it became almost clear with the lightest shade of magenta pink once completely dried.

In a third example, a composition was prepared by homogenously mixing the following in an enclosed stainless-steel tank: 91.3% DAITOSOL 5500GX by KOBO; 4.00% WIDE LASH by Sederma; 0.400 464004 Purple Carrot P150 by DDW The Color House; 2.00% butyle methacrylate by Sigma-Aldrich; 1.00 Cremophor RH 40 by BASF; 0.300 tocopheryl acetate by Lotioncrafter; and 1.00% Euxyle PE 9010 by Ashland. The composition has the following inactive ingredienets: acrylates/ethylhexyl acrylate copolymer, water, glycerin, butyl methacrylate, PEG-40 hydrogenated castor oil, tocopheryl acetate, panthenol, purple carrot extract, biotinoyl tripeptide-1, sodium dehydroacetate, phenoxyethanol, and ethylhexylglycerin.

The third example composition of the color changing cosmetic adhesive has an initial periwinkle color, an adhsesive or glue scent, and appears as a viscous liquid. The initial pH of the color changing cosmetic adhesive was 7.66, which was within an acceptable range of 7.16 to 8.16. The viscosity of the color changing cosmetic adhesive was 40,000 cP (s7/100 rpm), which was within an acceptable range of 39,000 to 41,000 cP. The density of the color changing cosmetic adhesive was 1.12, which was within an acceptable range of 1.06 to 1.18.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A color changing cosmetic adhesive for adhering a cosmetic item, comprising:
an adhesive formulation; and
a dye formulation distributed within the adhesive formulation;
wherein the dye formulation is configured to impart a first color to the adhesive formulation while the adhesive formulation is stored in a container; and
wherein the dye formulation is configured to impart a second color to the adhesive formulation after the adhesive formulation is applied to skin.

2. The color changing cosmetic adhesive of claim 1, wherein the cosmetic item is a false eyelash.

3. The color changing cosmetic adhesive of claim 2, wherein the adhesive formulation is applied to an eyelid.

4. The color changing cosmetic adhesive of claim 3, wherein the adhesive formulation is sufficiently cohesive to adhere the cosmetic item when the adhesive formulation cures to a threshold extent.

5. The color changing cosmetic adhesive of claim 4, wherein the adhesive formulation is configured to cure when the adhesive formulation is exposed to air humidity.

6. The color changing cosmetic adhesive of claim 5, wherein the adhesive formulation cures to the threshold extent necessary to adhere the cosmetic item when the adhesive formulation is exposed to air humidity for a threshold duration.

7. The color changing cosmetic adhesive of claim 6, wherein the threshold duration of the adhesive formulation depends on the pH level of the eyelid.

8. The color changing cosmetic adhesive of claim 7, wherein the adhesive formulation is tacky when cured to the threshold extent.

9. The color changing cosmetic adhesive of claim 1, wherein:
the adhesive formulation has an initial pH level when stored in the container; and
the dye formulation imparts the second color to the adhesive formulation when a current pH level of the adhesive formulation decreases from the initial pH level.

10. The color changing cosmetic adhesive of claim 9, wherein the current pH level of the adhesive formulation decreases from the initial pH level when applied to skin.

11. The color changing cosmetic adhesive of claim 10, wherein the initial pH level is between 7.5 and 7.7.

12. The color changing cosmetic adhesive of claim 11, wherein the current pH level of the adhesive formulation is between 5.4 and 7.5 when the dye formulation imparts the second color to the adhesive formulation.

13. The color changing cosmetic adhesive of claim 1, wherein:
the first color absorbs light in a first wavelength range; and
the second color absorbs light in a second wavelength range.

14. The color changing cosmetic adhesive of claim 13, wherein the first wavelength range is 570 to 620 nanometers.

15. The color changing cosmetic adhesive of claim 14, wherein the second wavelength range is 450 to 560 nanometers.

16. The color changing cosmetic adhesive of claim 1, wherein the dye formulation includes a vegetable-based ingredient.

17. The color changing cosmetic adhesive of claim 16, wherein the dye formulation includes a purple carrot extract.

18. The color changing cosmetic adhesive of claim 1, wherein the dye formulation includes a grain-based ingredient.

19. The color changing cosmetic adhesive of claim 18, wherein the dye formulation includes a purple rice extract.

20. The color changing cosmetic adhesive of claim 1, wherein the adhesive formulation comprises:
water;
a copolymer of acrylates and ethylhexyl acrylate;
glycerin; and
butyl methacrylate.
